(19) United States
(12) Patent Application Publication
(10) Pub. No.: US 2005/0131235 A1
(43) Pub. Date: Jun. 16, 2005

(54) Catalytic process for production of pyridine and picolines

(75) Inventors: Ashutosh Agarwal, Uttar Pradesh (IN); Pradeep Kumar Verma, Uttar Pradesh (IN); Kumar Samir Singh, Uttar Pradesh (IN); Praphulla Narahar Joshi, Maharashtra (IN); Gopal Moreshwar Chaphekar, Maharashtra (IN); Prashant Suresh Niphadkar, Maharashtra (IN); Rajiv Kumar, Maharashtra (IN)

Correspondence Address: NIXON & VANDERHYE, PC, 1100 N GLEBE ROAD, 8TH FLOOR, ARLINGTON, VA, 22201-4714, US

(73) Assignees: COUNCIL OF SCIENTIFIC AND INDUSTRIAL RESEARCH, New Delhi (IN); JUBILANT ORGANOSYS LIMITED, Uttar Pradesh (IN)

(21) Appl. No.: 10/731,440

(22) Filed: Dec. 10, 2003

(51) Int. Cl. C07D 213/08; C07D 213/14

(52) U.S. Cl. 546/250

(57) ABSTRACT

The present invention relates to a single step new catalytic process for the production of pyridine and picolines from a mixture of carbonyl compound and ammonia in the presence of zeolite catalyst with MFI topology containing Si and Zr and/or Sn as zeolite constituents in gas phase. The catalyst is preferably loaded with other metal ions such as lead, nickel, thallium or their mixture for increased yield. Present invention provides the novel use of above mentioned zeolite catalysts for the production of picolines for the first time, with improved yield of desired products picolines.

### FIELD OF THE INVENTION

The present invention relates to a catalytic process for the production of pyridine and picolines. More particularly it relates to a single step catalytic process for the production of pyridine and picolines by contacting a carbonyl compound such as an aldehyde represented by formaldehyde, acetaldehyde, propionaldehyde and/or a ketone such as acetone, propionone and the like with ammonia over porous solid catalyst(s) in gas phase aiming at high activity, selectivity and over all productivity.

### BACKGROUND OF THE INVENTION

Pyridine and picolines (where a methyl group, attached to the carbon ring, can be present at three different regio positions, with respect to ring nitrogen, such as 2-methyl pyridine or α-picoline, 3-methylpyridine or β-picoline and 4-methylpyridine or γ-picoline) are important intermediate compounds in the manufacture of agricultural chemicals (like herbicides and pesticides) and pharmaceuticals, and are also used as specific solvent in different industries like textile, polymer and pharmaceuticals.

Although, pyridine and picolines can be obtained as by-products in coal tar industry, due to small amount of pyridine and picolines present in coal tar the preferred method for producing pyridine and picolines is by chemical synthesis. Chemical method for the synthesis of these pyridines and picolines is based on a catalytic process where carbonyl compounds such as an aldehyde represented by formaldehyde, acetaldehyde, propionaldehyde and/or a ketone such as acetone, propionone and the like are reacted with ammonia in gas phase over a bed of solid catalyst such as amorphous silica-alumina (see for example U.S. Pat. No. 2,807,618) and crystalline aluminosilicates, which are commonly known as zeolites (see for example U.S. Pat. No. 5,994,550).

Alumino-silicate zeolites and their metallosilicate analogues are crystalline, microporous silica based materials having different framework structures. When a trivalent metal ion like B³⁺, Al³⁺, Fe³⁺, Ga³⁺, As³⁺ etc are incorporated in a crystalline silica network, a net negative charge is generated on the framework. This net negative charge is balanced by another extra framework charge compensating, ion-exchangeable cation. When proton is present as charge compensating cation then the zeolites behave as solid Brönsted acid. However, when a tetravalent metal ion is incorporated in a silica network then there is no net negative charge generated and the zeolite framework remains neutral without ion-exchange property. Although, such zeolite having certain tetravalent metal ions, particularly transition metal ions other than Si, with neutral framework do not exhibit proton donating Brönsted acidity, it is likely that these zeolites exhibit remarkable redox and Lewis acid characteristics depending upon the chemical nature of the incorporated metal ion other than Si.

Aiming to improve the overall yield of desired pyridine and picolines, various zeolite catalysts where frame work aluminum is replaced either fully or partially, by one or more cation(s) selected from divalent cations like Co²⁺ (see U.S. Pat. No. 6,281,362) trivalent metal ions like Fe³⁺ and/or Ga³⁺ (see U.S. Pat. No. 4,810,794) or tetravalent metal ions like Ti⁴⁺ (see U.S. Pat. No. 6,281,362), in the zeolite tetrahedral framework, commonly known as metallo-silicate analogues of their corresponding alumino-silicate zeolites, are also used as catalyst. For example, in U.S. Pat. No. 4,810,794 Shimizu et al. and in U.S. Pat. No. 5,952,258 Saitoh et al. have claimed the use of a zeolite having Si and B, Al, Fe, and/or Ga as zeolite constituent element, where an atomic ratio of Si to B, Al, Fe and/or Ga of 12 to 1000, as catalyst for producing pyridine and picolines. Among a large number of zeolites with different structure or topology used as catalyst, zeolite with MFI type topology, commonly known as ZSM-5, provides superior performance.

However, the main drawback of these catalysts was relatively low yields of desired pyridine or picolines and quick deactivation of the catalyst. In order to improve the yield of the main products (pyridine or picolines) and catalysts life, other metal ions selected from group I to XVII are deposited on the zeolite catalyst via post synthesis modification (see for example U.S. Pat. Nos. 4,810,794; 4,866,179, 5,994,550 and 6,281,362).

U.S. Pat. No. 6,281,362 Iwamoto teaches that when a catalyst comprising Ti and/or Co along with Silica as zeolite constituent, commonly known as titanium silicate and/or cobalt silicate having MFI or MEL (commonly known as pentasil structure) zeolite framework and preferably loaded with Pb, Tl etc., is contacted with an aldehyde or ketone and ammonia in gas phase in the temperature range of 300-700° C., the overall yield of picolines is improved substantially compared to where Al, Fe and/or Ga was used as zeolite constituent along with Silica. From above mentioned prior art methods for the production of pyridine and picolines, it can be construed that not only a physical factor such as zeolite structure, but also the different metal constituent present both in the zeolite framework (as zeolite constituent) and non-framework positions (loaded by conventional post synthesis treatment), known as chemical factors, significantly influence the activity, selectivity and overall productivity of the catalyst.

### OBJECTS OF THE INVENTION

The main object of the present invention is to provide a catalytic process for high throughput production of pyridine and picolines of pyridine and picolines in gas phase.

Another object of the present invention is to provide intrinsically highly active, selective, productive and stable catalyst for the production of pyridine and picolines in gas phase in the temperature at about 300 to 600° C.

### SUMMARY OF THE INVENTION

The inventors herein have extensively researched for a method that can produce pyridine and picolines in improved yields using a catalyst, which has not been used before for the production of pyridines and picolines, with improved activity and productivity. As a result, the present inventors have found that, when Si is partially replaced by Zr and/or Sn in pentasil (such as MFI framework) zeolite framework structure the activity, selectivity and above all the productivity of the catalyst is improved significantly.

Accordingly, the present invention provides a catalytic process for the production of pyridine and picolines which comprises contacting a mixture of a carbonyl compound and ammonia in the presence of zeolite catalyst with MFI topology in gas phase, condensing and separating the products.

In one embodiment of the invention, the contacting between the carbonyl compound and ammonia in the presence of the zeolite catalyst is carried out at a temperature in the range of 300-500° C., at a gas hourly space velocity in the range of 300 to 3000 h⁻¹ and pressure in the range of 1 to 10 atmosphere.

In another embodiment of the invention, the products obtained are purified by any conventional method.

In another embodiment of the invention, the carbonyl compound is selected from the group consisting of an aldehyde, a ketone and any mixture thereof.

In one embodiment of the invention, the aldehyde is an aliphatic aldehyde with 1 to 5 carbon atoms selected from the group consisting of formaldehyde, acetaldehyde, propionaldehyde and butyraldehyde.

In another embodiment of the invention, the ketone is an aliphatic ketone having 3 to 5 carbon atoms and selected from the group consisting of acetone, methyl ethyl ketone, and diethyl ketone.

In another embodiment of the invention, the carbonyl compound is an aldehyde selected from the group consisting of formaldehyde, acetaldehyde and propionaldehyde.

In another embodiment of the invention, the carbonyl compound is a ketone selected from acetone and propionone.

In another embodiment the catalyst has molecular formula 1 SiO₂:x MO₂, where M=Zr or Sn or a mixture thereof, and x is in the range of 0.002 and 0.05, with a crystal structure characterized by powder X-ray diffraction pattern as given in Table (1).

TABLE (1)

| | 2 theta, | Relative |
|---|---|---|
| No. | degree | intensity^{a} |
| 1 | 7.86 ± 0.05 | S |
| 2 | 8.78 ± 0.05 | MS |
| 3 | 13.18 ± 0.05 | W |
| 4 | 13.86 ± 0.05 | MW |
| 5 | 14.74 ± 0.05 | MW |
| 6 | 15.46 ± 0.05 | MW |
| 7 | 15.89 ± 0.05 | MW |
| 8 | 16.48 ± 0.05 | MW |
| 9 | 17.26 ± 0.05 | W |
| 10 | 17.64 ± 0.05 | W |
| 11 | 17.82 ± 0.05 | W |
| 12 | 19.22 ± 0.05 | W |
| 13 | 20.36 ± 0.05 | MW |
| 14 | 20.80 ± 0.05 | MW |
| 15 | 22.20 ± 0.05 | MW |
| 16 | 23.08 ± 0.05 | VS |
| 17 | 23.90 ± 0.05 | S |
| 18 | 24.40 ± 0.05 | MS |
| 19 | 25.69 ± 0.05 | MW |
| 20 | 25.89 ± 0.05 | W |
| 21 | 26.64 ± 0.05 | W |
| 22 | 27.42 ± 0.05 | W |
| 23 | 29.26 ± 0.05 | W |
| 24 | 29.90 ± 0.05 | MW |
| 25 | 45.10 ± 0.05 | W |
| 26 | 45.52 ± 0.05 | W |

^{a}R.I. = Relative Intensity,
VS = very strong,
S = strong,
M = medium,
W = weak

In another embodiment of the invention, the catalyst comprises a zeolite containing zirconium and/or tin and silicon as zeolite constituent elements wherein the atomic ratio of silicon to zirconium and/or tin is about 10 to about 500 and more preferably about 20 to about 100.

In another embodiment, the zeolite catalyst is loaded with a metal selected from the group consisting of lead, nickel, thallium and any mixture thereof using conventional impregnation method, where the metal loading is in the range of 3 and 12 wt %.

In yet another embodiment of the invention, the zeolite catalyst is in the form of a solid powder catalyst optionally mixed with inert binding substances selected from the group consisting of silica, alumina and any mixture thereof and shaped into extrudates or pellets as desired, dried and calcined or spray dried to obtain desired particle size, preferably in the range of 50-100 microns.

### DETAILED DESCRIPTION OF THE INVENTION

The present invention provides a new catalyst with high intrinsic catalytic activity and efficiency for producing pyridine and picolines, which comprises reacting in a gas-phase an aliphatic aldehyde, aliphatic ketone or mixture thereof with ammonia in the presence of a zeolite containing zirconium and/or tin along with silicon as zeolite constituent elements in which the atomic ratio of silicon to zirconium, silicon to tin or silicon to (zirconium+tin) is about 20 to about 500. These materials were prepared by suitably modifying the methods described in Europian Patent No.77,523 (1983) for zirconium-silicate MFI molecular sieve and N. K. Mal et al., J. Chem. Soc., Chem. Commun. 1933 (1994), so that zirconium silicate and tin silicate zeolites where Zr and/or Sn are the zeolite framework constituent can be obtained.

Another feature of the present invention is that the aliphatic aldehyde is preferably an aliphatic aldehyde or ketone having 1 to 5 carbon atoms. Examples thereof include aliphatic aldehydes such as formaldehyde, acetaldehyde, propionaldehyde, butylaldehyde and the like. The aliphatic ketone is preferably an aliphatic ketone having 3 to 5 carbon atoms. Examples thereof include acetone, methyl ethyl ketone, diethyl ketone and the like. As described above, a zeolite containing zirconium and/or tin and silicon as zeolite constituent elements in which the atomic ratio of silicon to zirconium or and/or tin is about 10 to about 500 and more preferably about 20 to about 100 is used as the catalyst in the reaction of the present invention. Hereinafter, the above-described zeolite, which is used as the catalyst in the present invention, is referred to as zirconium-silicate (Zr-Silicate), tin-silicate (Sn-Silicate) or zirconium-tin-silicate (Zr—Sn-Silicate) zeolite, herein after denoted as “zeolite catalyst”.

Still another, feature of the present invention is that the as-prepared zeolite catalyst is subjected to calcination at about 500-700° C. preferably in the presence of air or nitrogen or mixture thereof for about 6 to 24 hours to obtain organic free zeolite catalyst, which can optionally be subjected to 1 to 10 weight % aqueous solution of ammonium nitrate for ca. 1-4 hours at temperature at about 25 to 100° C., drying and calcining at about 500-700° C. in the presence of air or nitrogen or mixture thereof for about 6 to 24 hours.

Another feature of the present invention is that the zeolite catalyst is loaded with other metal like lead, nickel, thallium or mixtures thereof using conventional impregnation method.

Yet another feature of the present invention is that the mixture of an aldehyde or ketone or mixture thereof as mentioned above and ammonia is contacted with zeolite catalyst in a gas phase at a temperature at about 300 to 500° C. at gas space velocity in the range of 300 to 3000 h⁻¹ at the reaction pressure at 1 atmospheric or more. After the reaction, the pyridine and/or picolines coming out of the reactor in a gaseous stream can be condensed and separated from the unconverted reactants, if any, recovered and purified using conventional methods like distillation or alternatively the reaction products substantially containing the pyridine and/or picolines are dissolved in a solvent and distilled to recover the pyridine and/or picolines.

The present invention provides a catalytic process for the production of pyridine and picolines which comprises contacting a mixture of carbonyl compound and ammonia in the presence of zeolite catalyst with MFI topology in gas phase. The reaction is preferably carried out at a temperature ranging between 300-500° C., at gas space velocity in the range of 300 to 3000 h⁻¹ and pressure ranging between 1 to 10 atmosphere. The products obtained are condensed and separated by any conventional method.

The carbonyl compound can be an aldehyde represented by formaldehyde, acetaldehyde, propionaldehyde or a ketone such as acetone, propionone.

The catalyst used has molecular formula 1 SiO₂:x MO₂, where M=Zr or Sn or mixture thereof, and x may be in the range of 0.002 and 0.05, having crystal structure characterized by powder X-ray diffraction pattern as described in Table (1).

TABLE (1)

| | 2 theta, | Relative |
|---|---|---|
| No. | degree | intensity^{a} |
| 1 | 7.86 ± 0.05 | S |
| 2 | 8.78 ± 0.05 | MS |
| 3 | 13.18 ± 0.05 | W |
| 4 | 13.86 ± 0.05 | MW |
| 5 | 14.74 ± 0.05 | MW |
| 6 | 15.46 ± 0.05 | MW |
| 7 | 15.89 ± 0.05 | MW |
| 8 | 16.48 ± 0.05 | MW |
| 9 | 17.26 ± 0.05 | W |
| 10 | 17.64 ± 0.05 | W |
| 11 | 17.82 ± 0.05 | W |
| 12 | 19.22 ± 0.05 | W |
| 13 | 20.36 ± 0.05 | MW |
| 14 | 20.80 ± 0.05 | MW |
| 15 | 22.20 ± 0.05 | MW |
| 16 | 23.08 ± 0.05 | VS |
| 17 | 23.90 ± 0.05 | S |
| 18 | 24.40 ± 0.05 | MS |
| 19 | 25.69 ± 0.05 | MW |
| 20 | 25.89 ± 0.05 | W |
| 21 | 26.64 ± 0.05 | W |
| 22 | 27.42 ± 0.05 | W |
| 23 | 29.26 ± 0.05 | W |
| 24 | 29.90 ± 0.05 | MW |
| 25 | 45.10 ± 0.05 | W |
| 26 | 45.52 ± 0.05 | W |

^{a}R.I. = Relative Intensity,
VS = very strong,
S = strong,
M = medium,
W = weak

The zeolite catalyst is loaded with other metal such as lead, nickel, thallium or mixtures thereof using conventional impregnation method, where the metal loading may range between 3 and 12 wt %. The solid powder catalyst can be optionally mixed with inert binding substances like silica, alumina or mixture thereof and shaped in to extrudates or pallets as desired, dried and calcined or spray dried to obtain desired particle size, preferably in the range of 50-100 microns.

The process of the present invention is described herein below with examples, which are illustrative only and should not be construed to limit the scope of the present invention in any manner.

### EXAMPLE 1

This example illustrates the preparation of zirconium silicate molecular sieve. In a typical preparation 370 g of aqueous solution of tetra n-propyl ammonium hydroxide having 20% weight/weight (w/w) concentration was taken in a poly vinyl carbonate (PVC) container followed by the addition of 165 g of ethyl silicate (40 wt % silica) under vigorous stirring to the above solution over a period of about 20 minutes and the mixture was stirred for 2 hours. A solution of 17.2 g zirconium isopropoxide in 51 g of isopropanol was added to the above mixture over a period of 10 min. This mixture was again stirred for 1 hour. Then 190 g of deionised water was added and the resulting mixture was vigorously mixed for 1 hour. The pH of the gel was measured to be about 12.2. The gel was then transferred to a 2 liter autoclave. The temperature was raised to 170° C. and this temperature was maintained for 96 hours and then the contents were cooled to room temperature. The resulting slurry was centrifuged and the solid product thus obtained was washed with deionised water. The wet cake was dried for 4 hours at 120° C. followed by calcination at 540° C. for 16 hours in presence of air. The Si/Zr atomic ratio in the solid was 30 and the size of the cuboid shaped crystals was in the range of 0.6 and 1.0 micron. This catalyst is denoted as catalyst ZrS-1A.

### EXAMPLE 2

This example illustrates the preparation of zirconium silicate molecular sieve with smaller crystallites. In a typical preparation 370 g of aqueous solution of tetra n-propyl ammonium hydroxide having 20% weight/weight (w/w) concentration was taken in a poly vinyl carbonate (PVC) container followed by the addition of 230 g of tetraethyl orthosilicate under vigorous stirring to the above solution over a period of about 20 minutes and the mixture was stirred for 2 hours. A solution of 17.2 g zirconium isopropoxide in 51 g of isopropanol was added to the above mixture over a period of 10 min. This mixture was again stirred for 1 hour. Then 190 g of deionised water was added and the resulting mixture was vigorously mixed for 1 hour. The pH of the gel was measured to be about 12.2. The gel was then transferred to a 2 liter autoclave. The temperature was raised to 170° C. and this temperature was maintained for 96 hours and then the contents were cooled to room temperature. The resulting slurry was centrifuged and the solid product thus obtained was washed with deionised water. The wet cake was dried for 4 hours at 120° C. followed by calcination at 540° C. for 16 hours in presence of air. The Si/Zr atomic ratio in the solid was 30 and the size of the cuboid shaped crystallites was in the range of 0.2-0.5 micron. This sample is denoted as ZrS-1-B.

### EXAMPLE 3

This example illustrates the synthesis of tin silicate catalyst. In a typical preparation, 370 g of aqueous solution of tetra n-propyl-ammonium hydroxide 20% concentration w/w was taken in a PVC container followed by the addition 222 g of ethyl silicate (40 wt. % Silica) was added slowly but with vigorous stirring to the above solution over a period of 20 min and the mixture was stirred for 2 hours. 13.3 g of SnCl₄ in 50 g water was added to the above mixture over a period of 10 minutes and the mixture was again stirred for 1 hour. Then 145 g of deionised water was added and the resulting mixture was vigorously mixed for 1 hour. The pH of the gel was about 12. The gel was then transferred to an autoclave. The temperature was raised to 170° C. This temperature was maintained for 96 hours and then the contents were cooled. The resulting slurry was centrifuged and the solids are washed with deionised water. The wet cake was dried for 4 hours at 120° C. It was then calcined at 540° C. for 16 hours in the presence of air. The Si/Sn atomic ratio in the solid was 50 and the size of the cuboid shaped crystallites was in the range of 0.82-1.2 micron. This sample so obtained was denoted as Sn-S-1A.

### EXAMPLE 4

This example illustrates the synthesis of tin silicate catalyst with smaller particles. In a typical preparation, 370 g of aqueous solution of tetra n-propyl-ammonium hydroxide 20% concentration w/w was taken in a PVC container followed by the addition 310 g of tetra ethyl orthosilicate was added slowly but with vigorous stirring to the above solution over a period of 20 min and the mixture was stirred for 2 hours. 13.3 g of SnCl₄ in 50 g water was added to the above mixture over a period of 10 minutes and the mixture was again stirred for 1 hour. Then 145 g of deionised water was added and the resulting mixture was vigorously mixed for 1 hour. The pH of the gel was about 12. The gel was then transferred to an autoclave. The temperature was raised to 170° C. This temperature was maintained for 96 hours and then the contents were cooled. The resulting slurry was centrifuged and the solids are washed with deionised water. The wet cake was dried for 4 hours at 120° C. It was then calcined at 540° C. for 16 hours in the presence of air. The Si/Sn atomic ratio in the solid was 50 and the crystallite size of this sample was between 0.3-0.6 micron. This sample so obtained was denoted as Sn-S-1B.

### EXAMPLE 5

This example illustrates the loading of metal such as lead on calcined ZrS-1 or SnS-1 catalyst using impregnation/kneading method. In a typical method, 250 g of catalyst was contacted under stirring condition with a solution containing 31 g lead nitrate in 350 g water. The whole slurry was then evaporated to dryness and the solid thus obtained was dried at 120° C. for 5 hours, followed by calcination at 550° C. in the presence of air for 5 hours.

### EXAMPLE 6

This example illustrates the method for carrying out catalytic reaction. Catalyst ZrS-1A and ZrS-1B prepared by kneading method with 7.1 wt % loading of lead and containing 17 wt. % binder were pelletized and evaluated in SS 316 reactor tube with 31 mm I.D and 750 cc catalyst capacity, down flow, fixed bed reactor. A mixed gas of acetaldehyde and ammonia optionally along with water/steam as diluent were pre-heated at 275° C. and the vapours allowed to pass over the catalyst bed kept at isothermal condition. Catalyst bed temperature was maintained between 395±5° C. The exit gases containing the pyridine bases were condensed and analyzed for the components. Finally, the resultant condensate or the reaction mass is extracted with a solvent and fractionated to recover the pyridine bases. After prolonged reaction when the catalyst gets de-activated then it is regenerated by passing air at 500-550° C. Preferably, air is diluted with nitrogen during the regeneration of the catalyst. The results obtained with different catalysts are given in Table (2).

### EXAMPLE 7

This example illustrates the method for carrying out catalytic reaction. Catalyst SnS-1A and SnS-1B prepared by kneading method with 7.1 wt % loading of lead and containing 17 wt. % binder were pelletized and evaluated in SS 316 reactor tube with 31 mm I.D and 750 cc catalyst capacity, down flow, fixed bed reactor. A mixed gas of acetaldehyde and ammonia optionally along with water/steam as diluent were pre-heated at 275° C. and the vapours allowed to pass over the catalyst bed kept at isothermal condition. Catalyst bed temperature was maintained between 395±5° C. The exit gases containing the pyridine bases were condensed and analyzed for the components. Finally, the resultant condensate or the reaction mass is extracted with a solvent and fractionated to recover the pyridine bases. After prolonged reaction when the catalyst gets de-activated then it is regenerated by passing air at 500-550° C. Preferably, air is diluted with nitrogen during the regeneration of the catalyst. The results obtained are given in Table (3).

### EXAMPLE 8

This example compares the catalytic performance of the catalysts used in present invention with that of prior-art catalysts. The reaction conditions mentioned in example 6 or 7 were used except the catalysts. Two catalyst systems adapted from Table 4 (example 3 and 4) of U.S. Pat. No. 6,281,362 were chosen, as these examples provided the best yields of picolines reported in prior catalytic process, for comparative purpose with the performance of the catalytic process of the present invention (example 6 and 7). The total yield of picolines (desired products) obtained over prior art catalysts 7% Pb—Ti/Si (Table 4, example 3 of U.S. Pat. No. 6,281,362) and 3% Pb—Co/Ti (Table 4, example 4 of U.S. Pat. No. 6,281,362) was 69.1 and 71.7%, respectively, to be compared to the total picoline yield of 74.9% and 75.7 obtained over 7.1% Pb—Zr-S-1B (example 6) and 7.1Pb—Sn-S-1B (example 7) catalysts of the present invention. The corresponding picoline yield obtained over the prior art catalyst (7% Pb—Si/Ti) prepared according to example 3 of U.S. Pat. No. 6,281,362 by us under otherwise comparable reaction conditions was 69.2%, which is quite comparable with the reported corresponding value of 69.1%. Above results show that the new catalytic process of the present invention provides higher yields of picoline vis-à-vis prior art method.

TABLE 2
Comparison of catalytic performance of different Zr—S-1 catalysts

| | | 7.1% Pb/ | | 7.1% |
|---|---|---|---|---|
| Parameters | ZrS-1A | ZrS-1A | ZrS-1B | Pb/ZrS-1B |
| Alpha picoline % | 29.1 | 44.7 | 36.0 | 50.6 |
| Gamma picoline % | 15.2 | 22.3 | 18.0 | 25.3 |
| Pyridine % | 1.1 | 1.2 | 1.0 | 1.1 |
| Total yield % | 45.4 | 68.2 | 55.0 | 76.0 |
| Conversion % | 93.0 | 98.0 | 94.0 | 98.0 |
| Selectivity % | 48.8 | 69.6 | 58.5 | 77.5 |
| Alpha to Gamma | 1.9:1 | 2:1 | 2.0:1 | 2.0:1 |
| picoline ratio | | | | |
| Space velocity^{h-1} | 425 | 625 | 575 | 675 |

TABLE 3
Comparison of catalytic performance of different Sn—S-1 catalysts

| | | 7.1% Pb/SnS- | | 7.1% Pb/SnS- |
|---|---|---|---|---|
| Parameters | SnS-1A | 1A | SnS-1B | 1B |
| Alpha picoline % | 29.5 | 46.6 | 38.0 | 53.4 |
| Gamma picoline % | 14.1 | 19.4 | 19.0 | 22.3 |
| Pyridine % | 1.0 | 1.1 | 1.0 | 1.0 |
| Total yield % | 44.6 | 67.1 | 58.0 | 76.7 |
| Conversion % | 93.5 | 97.0 | 94.0 | 98.2 |
| Selectivity % | 47.7 | 70.0 | 61.7 | 78.1 |
| Alpha to Gamma | 2.1:1 | 2.4:1 | 2.0:1 | 2.4:1 |
| picoline ratio | | | | |
| Space velocity^{h-1} | 475 | 775 | 675 | 850 |

1. A catalytic process for the production of pyridine and picolines which comprises contacting a mixture of a carbonyl compound and ammonia in the presence of zeolite catalyst with MFI topology in gas phase, condensing and separating the products.

2. A process as claimed in claim 1 wherein the contacting between the carbonyl compound and ammonia in the presence of the zeolite catalyst is carried out at a temperature in the range of 300-500° C., at a gas hourly space velocity in the range of 300 to 3000 h⁻¹ and pressure in the range of 1 to 10 atmosphere.

3. A process as claimed in claim 1 wherein the products obtained are separated and purified.

4. A process as claimed in claim 1 wherein the carbonyl compound is selected from the group consisting of an aldehyde, a ketone and any mixture thereof.

5. A process as claimed in claim 4 wherein the aldehyde is an aliphatic aldehyde with 1 to 5 carbon atoms selected from the group consisting of formaldehyde, acetaldehyde, propionaldehyde and butyraldehyde.

6. A process as claimed in claim 4 wherein the ketone is an aliphatic ketone having 3 to 5 carbon atoms and selected from the group consisting of acetone, methyl ethyl ketone, and diethyl ketone.

7. A process as claimed in claim 1 wherein the catalyst has molecular formula 1SiO₂:x MO₂, where M=Zr or Sn or a mixture thereof, and x is in the range of 0.002 and 0.05, with a crystal structure characterized by powder X-ray diffraction pattern as given in Table (1).
TABLE (1)

| | 2 theta, | Relative |
|---|---|---|
| No. | degree | intensity^{a} |
| 1 | 7.86 ± 0.05 | S |
| 2 | 8.78 ± 0.05 | MS |
| 3 | 13.18 ± 0.05 | W |
| 4 | 13.86 ± 0.05 | MW |
| 5 | 14.74 ± 0.05 | MW |
| 6 | 15.46 ± 0.05 | MW |
| 7 | 15.89 ± 0.05 | MW |
| 8 | 16.48 ± 0.05 | MW |
| 9 | 17.26 ± 0.05 | W |
| 10 | 17.64 ± 0.05 | W |
| 11 | 17.82 ± 0.05 | W |
| 12 | 19.22 ± 0.05 | W |
| 13 | 20.36 ± 0.05 | MW |
| 14 | 20.80 ± 0.05 | MW |
| 15 | 22.20 ± 0.05 | MW |
| 16 | 23.08 ± 0.05 | VS |
| 17 | 23.90 ± 0.05 | S |
| 18 | 24.40 ± 0.05 | MS |
| 19 | 25.69 ± 0.05 | MW |
| 20 | 25.89 ± 0.05 | W |
| 21 | 26.64 ± 0.05 | W |
| 22 | 27.42 ± 0.05 | W |
| 23 | 29.26 ± 0.05 | W |
| 24 | 29.90 ± 0.05 | MW |
| 25 | 45.10 ± 0.05 | W |
| 26 | 45.52 ± 0.05 | W |

^{a}R.I. = Relative Intensity,
VS = very strong,
S = strong,
M = medium,
W = weak

8. A process as claimed in claim 1 wherein the catalyst comprises a zeolite containing zirconium and/or tin and silicon as zeolite constituent elements wherein the atomic ratio of silicon to zirconium and/or tin is about 10 to about 500 and more preferably about 20 to about 100.

9. A process as claimed in claim 1 wherein the zeolite catalyst is loaded with a metal selected from the group consisting of lead, nickel, thallium and any mixture thereof using conventional impregnation method, where the metal loading is in the range of 3 and 12 wt %.

10. A process as claimed in claim 1 wherein the zeolite catalyst is in the form of a solid powder catalyst.

11. A process as claimed in claim 10 wherein the zeolite catalyst is mixed with inert binding substances selected from the group consisting of silica, alumina and any mixture thereof and shaped into extrudates or pellets as desired, dried and calcined or spray dried to a particle size in the range of 50-100 microns.
